(19) United States
(12) Patent Application Publication
(10) Pub. No.: US 2025/0170514 A1
(43) Pub. Date: May 29, 2025

(54) CONTAMINANT PROCESSING DEVICE

(71) Applicant: SEMES CO., LTD., Cheonan-si (KR)

(72) Inventor: Seung Chan LEE, Gyeonggi-do (KR)

(73) Assignee: SEMES CO., LTD., Cheonan-si (KR)

(21) Appl. No.: 18/921,392

(22) Filed: Oct. 21, 2024

(30) Foreign Application Priority Data
Nov. 27, 2023 (KR) 10-2023-0166240

(51) Int. Cl. B01D 46/44; B01D 46/46; G01N 15/06; G01N 33/00

(52) U.S. Cl. CPC B01D 46/442; B01D 46/46; G01N 15/06; G01N 33/0031; B01D 2279/51

(57) ABSTRACT

There is provided a contaminant processing device that purifies and measures particles or chemical gases generated in a semiconductor line. The contaminant processing device includes a body unit including different first surface and second surface, a driving unit installed in the body unit and configured to drive inside a semiconductor line, a position sensor installed in the body unit and configured to sense a position within the semiconductor line, a purification unit installed in the body unit and configured to penetrate the first surface and the second surface, wherein the purification unit includes a flow path disposed between the first surface and the second surface, a chemical gas detection sensor installed in the flow path, measuring a chemical gas entering through the first surface and a controller that receives a concentration of the chemical gas from the chemical gas detection sensor, receives position data from the position sensor, and checks the concentration of the chemical gas according to a position within the semiconductor line.

### CROSS-REFERENCE TO RELATED APPLICATION

This application claims priority from Korean Patent Application No. 10-2023-0166240 filed on Nov. 27, 2023 in the Korean Intellectual Property Office, and all the benefits accruing therefrom under 35 U.S.C. 119, the contents of which in its entirety are herein incorporated by reference.

### BACKGROUND

### Technical Field

The present disclosure relates to a contaminant processing device.

### Description of the Related Art

In general, a semiconductor or display device may be manufactured by repeatedly performing a series of manufacturing processes on a substrate such as a silicon wafer or a glass substrate. For example, manufacturing processes such as deposition, photo etch, oxidation, ion implantation, and cleaning may be selectively and/or repeatedly performed to form circuit patterns on the substrate.

Such manufacturing processes may be performed in a clean room. When the manufacturing processes are performed, particles or hazardous chemical gases may be generated in the clean room. Since particles or hazardous chemical gases have a significant impact on the manufacturing process, it is necessary to treat these contaminants to control contamination.

Aspects and features of embodiments of the present disclosure are to provide a contaminant processing device that purifies and measures particles or chemical gases generated in a semiconductor line.

Another Aspects and features of embodiments of the present disclosure are to provide a contaminant processing method that purifies and measures particles or chemical gases generated in a semiconductor line.

According to some aspects of the disclosure, there is provided a contaminant processing device comprising a body unit including different first surface and second surface, a driving unit installed in the body unit and configured to drive inside a semiconductor line, a position sensor installed in the body unit and configured to sense a position within the semiconductor line, a purification unit installed in the body unit and configured to penetrate the first surface and the second surface, wherein the purification unit includes a flow path disposed between the first surface and the second surface, a chemical gas detection sensor installed in the flow path, measuring a chemical gas entering through the first surface and a controller that receives a concentration of the chemical gas from the chemical gas detection sensor, receives position data from the position sensor, and checks the concentration of the chemical gas according to a position within the semiconductor line.

According to some aspects of the disclosure, there is provided a contaminant processing device comprising a purification unit including a suction fan installed in a first area and sucking in particles and chemical gases, a collection unit installed in a second area and filtering the particles and the chemical gases, a particle sensor and a chemical gas detection sensor installed in a third area between the first area and the second area, wherein the particle sensor measures the particles, and the chemical gas detection sensor measures the chemical gases, a flow path disposed in the third area, a body unit connected to the purification unit and including first surface and second surface, a driving unit installed in the body unit and configured to drive inside the semiconductor line, a position sensor installed in the body unit and sensing a position within the semiconductor line and a controller that receives a concentration of the particles from the particle sensor, receives a concentration of the chemical gas from the chemical gas detection sensor, and checks concentration of the particles and the chemical gas according to the position within the semiconductor line.

According to some aspects of the disclosure, there is provided a contaminant processing device comprising a body unit including different first surface and second surface, a driving unit installed in the body unit and configured to drive inside a semiconductor line, a position sensor installed in the body unit and configured to sense a position within the semiconductor line, a purification unit installed in the body unit and configured to penetrate the first surface and the second surface, wherein the purification includes a suction fan installed in a first area and sucking in particles and chemical gases, a collection unit installed in a second area and filtering the particles and the chemical gases, a flow path installed in a third area between the first area and the second area, a particle sensor for measuring the particle and a chemical gas detection sensor for measuring the chemical gas wherein the particle sensor and the chemical gas detection sensor installed in the flow path, an image processing unit receiving data from the position sensor, the particle sensor, and the chemical gas detection sensor to form a map of the semiconductor line and a controller that receives a concentration of the particles from the particle sensor, receives a concentration of the chemical gas from the chemical gas detection sensor, and a position of the particle and a position of the chemical gas from the position sensor, wherein the flow path includes one side, a center, and the other side, and wherein a width of the flow path gradually decreases from the one side to the center, and gradually increases from the center to the other side, wherein the semiconductor line includes first zone and second zone, wherein the particle sensor and the chemical gas detection sensor measure the concentration of the particles and the concentration of the chemical gas present in the first zone and the second zone, wherein the position sensor senses the positions of the particles and the chemical gas, wherein the image processing unit forms a first map showing the positions of the particles and the chemical gas present in the first zone and the second zone.

The technical problems of the present disclosure are not limited to the above-mentioned technical problems, and other technical problems not mentioned will be clearly understood by those skilled in the art from the description below.

### BRIEF DESCRIPTION OF THE DRAWINGS

FIGS. 1 to 5 are drawings illustrating a contaminant processing device according to some embodiments of the present disclosure.

FIGS. 6 and 7 are drawings to illustrate contaminant processing operation of a contaminant processing device according to some embodiments of the present disclosure.

FIGS. 8 and 9 are drawings to illustrate a contaminant processing device according to some embodiments of the present disclosure.

FIG. 10 is a drawing to illustrate a contaminant processing device according to some embodiments of the present disclosure.

FIG. 11 is a drawing to illustrate a contaminant processing device according to some embodiments of the present disclosure.

FIG. 12 is a drawing to illustrate a contaminant processing method according to some embodiments of the present disclosure.

### DETAILED DESCRIPTION

In this specification, although the terms “first,” “second,” and the like are used to describe various elements or components, these elements or components are not limited by these terms. These terms are only used to distinguish one element or component from another element or component. Therefore, the first element or component mentioned below may also be the second element or component within the technical concept of the present disclosure.

Hereinafter, embodiments of the present disclosure will be described in detail with reference to the attached drawings. Identical components in the drawings are designated by the same reference numerals, and redundant descriptions thereof will be omitted.

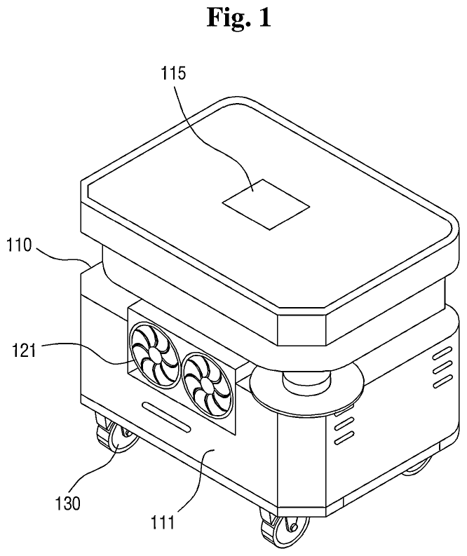

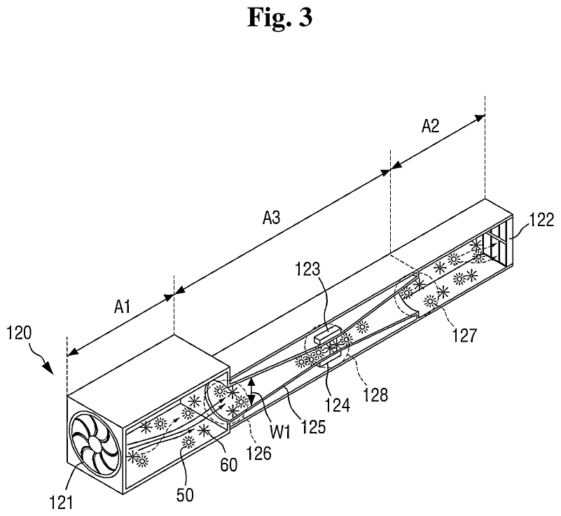

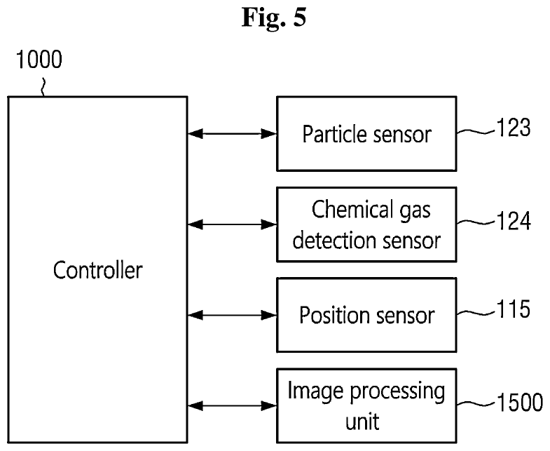

FIGS. 1 to 5 are drawings illustrating a contaminant processing device according to some embodiments of the present disclosure.

Referring to FIGS. 1 to 5, the contaminant processing device may include a body unit 110, a purification unit 120, a driving unit 130, a position sensor 115, an image processing unit 1500, and a controller 1000.

The body unit 110 may include a first surface 111 and a second surface 112 opposite to the first surface 111. The body unit 110 may include a plurality of surfaces. The body unit 110 may have an empty space inside. In FIG. 1, the body unit 110 is illustrated as a cube or a rectangular but is not limited thereto.

The body unit 110 may be directly or indirectly connected to the purification unit 120, the driving unit 130, the position sensor 115, the controller 1000, and the image processing unit 1500, which will be described later. The body unit 110 may be a body of the contaminant processing device.

The purification unit 120 may be coupled to the body unit 110. The purification unit 120 may be installed in the internal space of the body unit 110. The first surface 111 and the second surface 112 of the body unit 110 may be penetrated by the purification unit 120.

The purification unit 120 may include a first area A1, a second area A2, and a third area A3. The purification unit 120 may be formed to be extended in a horizontal direction. The first area A1, the second area A2, and the third area A3 of the purification unit 120 may be disposed in one direction. The third area A3 may be between the first area A1 and the second area A2.

The first area A1 may be disposed closer to the first surface 111 of the body unit 110 than the second area A2. The second area A2 may be disposed closer to the second surface 112 of the body unit 110 than the first area A1.

The purification unit 120 may include a flow path 125, a suction fan 121, a collection unit 122, a chemical gas detection sensor 124, and a particle sensor 123.

The flow path 125 may be disposed between the first surface 111 and the second surface 112 of the body unit 110. The flow path 125 may be installed in the third area A3 of the purification unit 120. The flow path 125 may have an elongated cylindrical shape.

The flow path 125 may include one side 126, a center 128, and the other side 127. The one side 126 may be a boundary between the first area A1 and the third area A3. The center 128 may be between the one side 126 and the other side 127. The other side 127 may be a boundary between the second area A2 and the third area A3.

The first area A1 may be disposed between one side 126 of the flow path 125 and the first surface 111 of the body unit 110. The first area A1 may have a width greater than the maximum width of the flow path 125.

The second area A2 may be disposed between the other side 127 of the flow path 125 and the second surface 112 of the body unit 110. The second area A2 may have a width greater than the maximum width of the flow path 125.

The width W1 of the flow path 125 may gradually decrease from one side 126 towards the center 128. The width W1 of the flow path 125 may gradually increase from the center 128 towards the other side 127.

The width W1 of the flow path 125 may not be constant along the direction from one side 126 to the other side 127. The width of the center 128 may be smaller than the width of the one side 126. The width of the center 128 may be smaller than the width of the other side 127.

The chemical gas detection sensor 124 may be installed in the flow path 125 installed in the third area A3 of the purification unit 120. The chemical gas detection sensor 124 may be installed in the center 128 of the flow path 125 but is not limited thereto. The chemical gas detection sensor 124 may be installed in the area with the smallest width in the flow path 125.

The chemical gas 50 sucked by the suction fan 121 to be described later may be measured by the chemical gas detection sensor 124. The concentration of the chemical gas 50 may be measured by the chemical gas detection sensor 124.

The particle sensor 123 may be installed in the flow path 125 installed in the third area A3 of the purification unit 120. The particle sensor 123 may be installed in the center 128 of the flow path 125 but is not limited thereto. The particle sensor 123 may be installed in the area with the smallest width in the flow path 125.

The particle sensor 123 may measure the particles 60 being sucked by the suction fan 121. The particle sensor 123 may measure the concentration of the particles 60.

The suction fan 121 may be installed in the first area A1 of the purification unit 120. The suction fan 121 may be installed at the end of the first area A1 but is not limited thereto. The suction fan 121 may be formed on one side 126 of the flow path 125.

The suction fan 121 may be installed on the first surface 111 of the body unit 110. The center of the first surface 111 of the body unit 110 may be opened so that the suction fan 121 may be exposed.

The suction fan 121 may be disposed on the same plane as the first surface 111 of the body unit 110 but is not limited thereto. The suction fan 121 may protrude from the first surface 111 of the body unit 110.

The suction fan 121 may include a single fan or a plurality of fans.

The suction fan 121 may include a motor. The suction fan 121 may suck in air using the motor. The suction fan 121 may suck in particles 60 or chemical gas 50. When the suction fan 121 sucks in air, the air may contain particles 60 or chemical gas 50. The suction fan 121 may move air from the first area A1 to the third area A3 and the second area A2.

When the suction fan 121 sucks air containing particles 60 and chemical gas 50, the particles 60 and chemical gas 50 may move from the first area A1 to the third area A3.

The chemical gas 50 may be measured by the chemical gas detection sensor 124 installed in the third area A3. The particles 60 may be measured by the particle sensor 123 installed in the third area A3.

The collection unit 122 may be installed in the second area A2 of the purification unit 120. The collection unit 122 may be installed at the end of the second area A2 but is not limited thereto. The collection unit 122 may be installed on the other side 127 of the flow path 125.

The collection unit 122 may be installed on the second surface 112 of the body unit 110. The center of the second surface 112 of the body unit 110 is open so that the collection unit 122 may be exposed. The collection unit 122 may be disposed on the same plane as the second surface 112 of the body unit 110 but is not limited thereto. The collection unit 122 may protrude from the second surface 112 of the body unit 110.

The chemical gas 50 may be sucked by the suction fan 121 and moved to the first area A1, the third area A3, and the second area A2 and filtered by the collection unit 122. That is, the chemical gas 50 may be captured by the collection unit 122.

The particles 60 may be sucked by the suction fan 121 and moved to the first area A1, the third area A3, and the second area A2 and filtered by the collection unit 122. That is, the particles 60 may be captured by the collection unit 122.

The collection unit 122 may be a fan filter unit FFU.

The driving unit 130 may be installed in the body unit 110. For example, the driving unit 130 may be installed on a lower portion of the body unit 110.

The driving unit 130 may include a wheel and a driving motor, and the like. The wheel may be installed on the lower portion of the body unit 110. The driving unit 130 may be moved by rotating the wheel by the driving motor. The driving unit 130 may drive inside the semiconductor line.

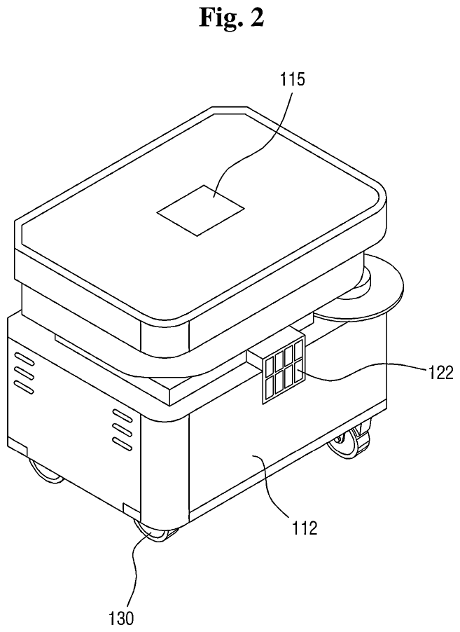

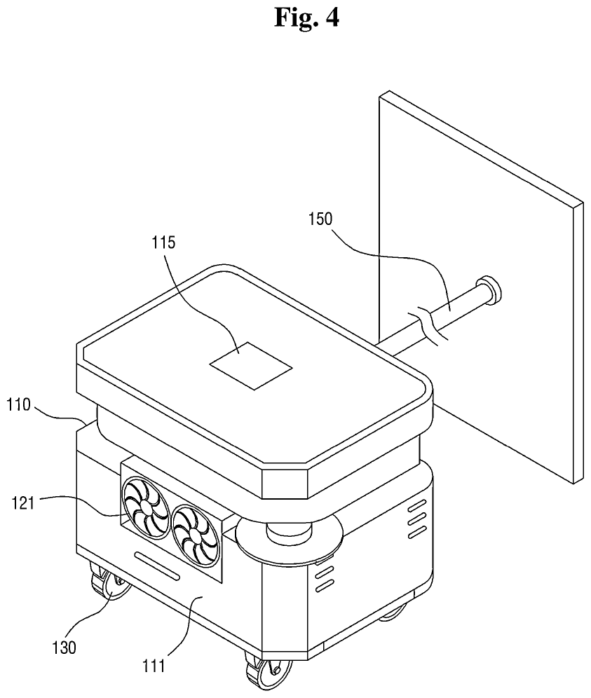

The position sensor 115 may be installed in the body unit 110. In FIG. 1, FIG. 2, and FIG. 4, the position sensor 115 is shown as being installed on an upper surface of the body unit 110 but is not limited thereto.

The position sensor 115 may sense the position of the contaminant processing device. For example, the position sensor 115 may sense the position of the contaminant processing device disposed inside the semiconductor line.

The position of the chemical gas 50 or the position of the particle 60 may be detected by the position sensor 115. When the chemical gas 50 and the particle 60 are measured by the chemical gas detection sensor 124 and the particle sensor 123, the positions of the chemical gas 50 and the particle 60 may be sensed by the position sensor 115.

An exhaust device 150 may be installed inside the semiconductor line. The exhaust device 150 may include a duct and a suction unit.

A contaminant processing device may be connected to the exhaust device 150. The contaminant processing device may be moved to the exhaust device 150 so that the chemical gases 50 and particles 60 filtered by the capture unit 122 may be removed through the exhaust device 150.

The chemical gas 50 and particles 60 filtered by the collection unit 122 may be suctioned by the suction unit and discharged to the outside through a duct.

The controller 1000 may control at least one of the particle sensor 123, the chemical gas detection sensor 124, the position sensor 115, and the image processing unit 1500.

The controller 1000 may be provided with a concentration of particles 60 from the particle sensor 123. The controller 1000 may be provided a concentration of the chemical gas 50 from the chemical gas detection sensor 124.

The controller 1000 may be provided with location data from the position sensor 115. The controller 1000 may be provided with a location of the contaminant processing device from the location sensor 115.

The controller 1000 may check the concentration of the chemical gas 50 and the concentration of the particles 60 based on the location in the semiconductor line.

The controller 1000 may transmit information on the concentration of the particle 60, information on the concentration of the chemical gas 50, and location data of the particle 60 and the chemical gas 50 to the image processing unit 1500.

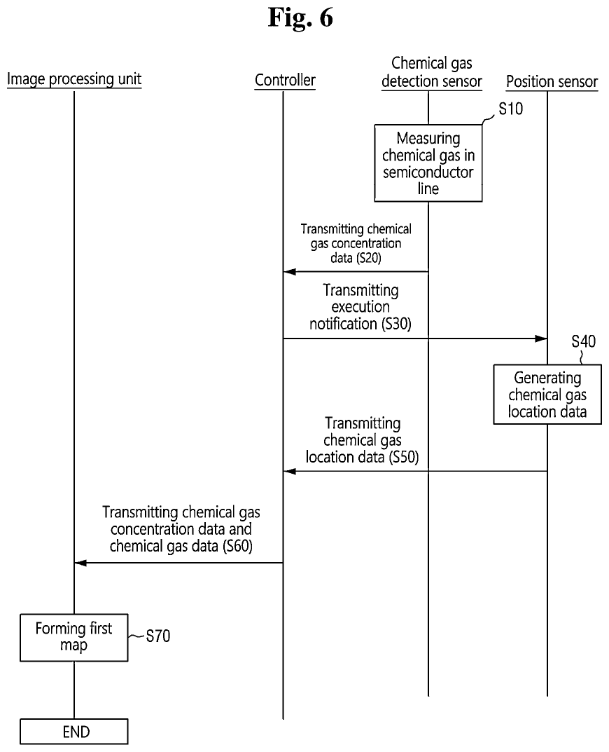

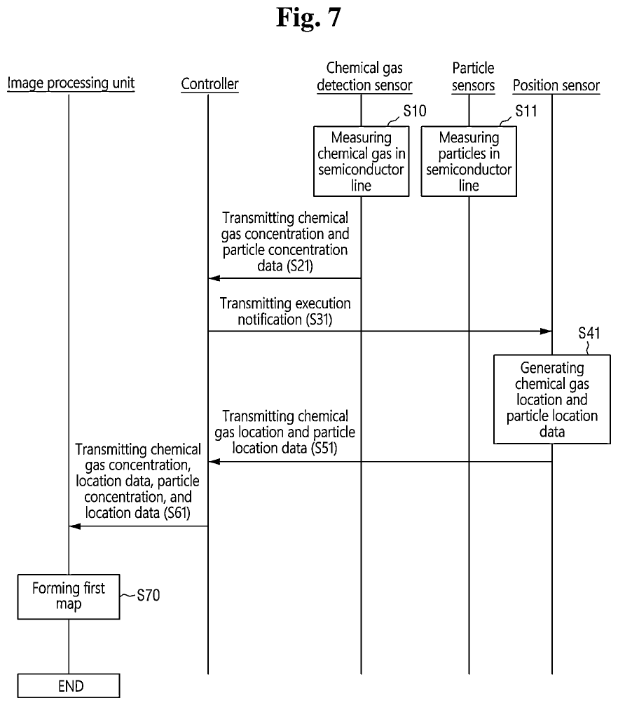

FIGS. 6 and 7 are drawings to illustrate contaminant processing operation of a contaminant processing device according to some embodiments of the present disclosure.

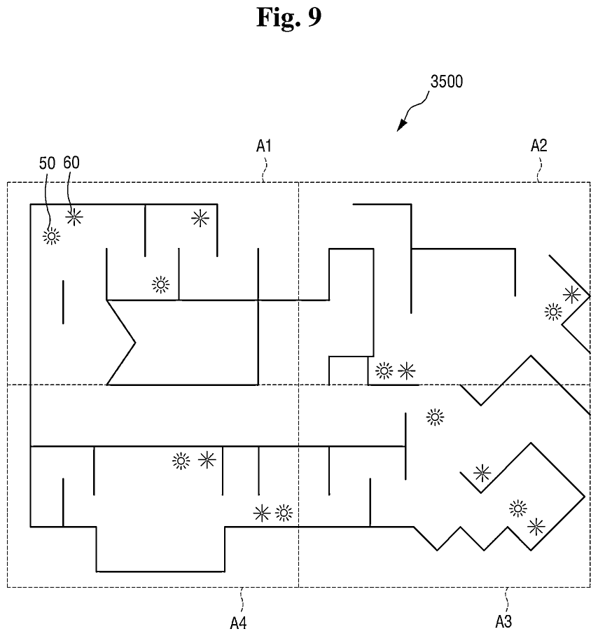

FIG. 6 illustrates a process in which a chemical gas 50 is measured by a chemical gas detection sensor 124, a location of the chemical gas 50 is measured by a location sensor 115, and a first map (3500 in FIG. 9) showing the chemical gas 50 is formed by an image processing unit 1500.

Referring to FIGS. 1 to 5 and FIG. 6, a chemical gas present in a semiconductor line is measured by a chemical gas detection sensor (S10).

The chemical gas 50 may be measured by a chemical gas detection sensor 124 installed in a flow path 125 of the purification unit 120 when the suction fan 121 sucks in the chemical gas 50.

Subsequently, the chemical gas detection sensor transmits the chemical gas 50 concentration data to a controller (S20).

The controller 1000 receives the chemical gas 50 concentration data from the chemical gas detection sensor 124 and may determine whether the concentration of the chemical gas 50 is high or low. For example, the controller 1000 may determine whether the chemical gas 50 concentration is higher or lower than a user-set threshold.

Next, if the chemical gas concentration is higher than the reference value, the controller transmits an execution notification to the position sensor (S30).

Then, the position sensor generates position data of the chemical gas (S40).

The position of the chemical gas 50 measured by the chemical gas detection sensor 124 may be sensed by the position sensor 115. Through this, the position of the contaminant processing device may be sensed.

Next, the position sensor transmits the chemical gas position data to the controller (S50).

Then, the controller transmits the chemical gas concentration data and the chemical gas position data to the image processing unit (S60).

Next, the image processing unit forms a first map based on the chemical gas concentration data and chemical gas location data provided from the controller (S70).

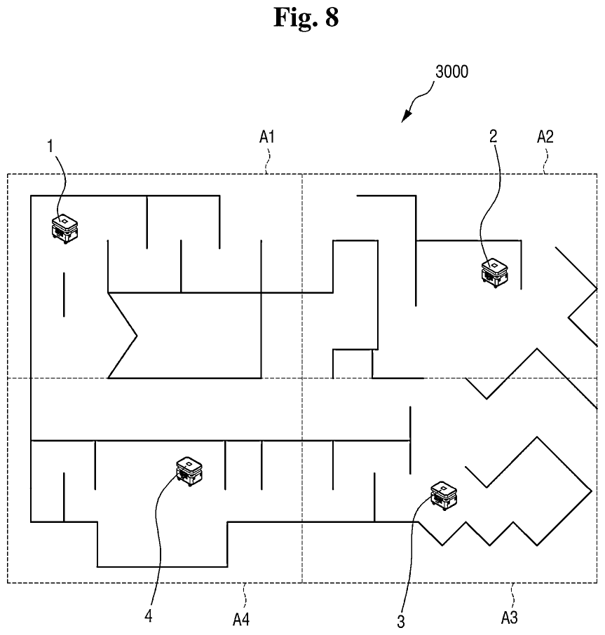

The first map (3500 of FIG. 9) may be a map of a semiconductor line. The first map 3500 may be a map showing the concentration and location of a chemical gas 50. A description of the first map 3500 will be described in more detail with reference to FIGS. 8 and 9.

FIG. 7 illustrates a process in which a chemical gas 50 and a particle 60 are measured by a chemical gas detection sensor 124 and a particle sensor 123, the location of the chemical gas 50 and the location of the particle 60 are measured by a location sensor 115, and a first map (3500 in FIG. 9) displaying the chemical gas 50 and the particle 60 is formed by the image processing unit 1500.

Referring to FIGS. 1 to 5 and 7, chemical gas is measured in a semiconductor line by a chemical gas detection sensor S10, and particles in a semiconductor line are measured by a particle sensor (S11).

The chemical gas 50 and the particles 60 may be measured by the chemical gas detection sensor 124 and the particle sensor 123 installed in the flow path 125 of the purification unit 120.

Then, the chemical gas detection sensor and the particle sensor transmit chemical gas concentration and particle concentration data to the controller (S21).

The controller 1000 receives the chemical gas 50 concentration and the particle 60 concentration from the chemical gas detection sensor 124 and the particle sensor 123 and may determine whether the concentrations are high or low. For example, it can be determined whether the chemical gas 50 concentration is higher or lower than a reference value set by a user. Also, it can be determined whether the particle 60 concentration is higher or lower than a user-set threshold.

If the chemical gas concentration and particle concentration are higher than the reference value, the controller transmits an execution notification to a position sensor (S31).

Then, the position sensor generates chemical gas position and particle position data (S41).

The position of the chemical gas 50 measured by the chemical gas detection sensor 124 and the position of the particle 60 measured by the particle sensor 123 may be sensed by the position sensor 115. Through this, the position of the contaminant processing device may be sensed.

Next, the position sensor transmits the chemical gas and particle position data to the controller (S51).

Then, the controller transmits the chemical gas concentration and position data and the particle concentration and position data to the image processing unit (S61).

Then, the image processing unit forms a first map based on the chemical gas concentration and position data and the particle concentration and position data provided from the controller (S70).

FIGS. 8 and 9 are drawings to illustrate a contaminant processing device according to some embodiments of the present disclosure.

Referring to FIG. 8, in some embodiments, the contaminant processing device may travel the semiconductor line 3000. FIG. 8 may be a map of the semiconductor line 3000.

The semiconductor line 3000 may include a first zone A1, a second zone A2, a third zone A3, and a fourth zone A4. Although not shown, semiconductor facility may be installed in each of the zones A1, A2, A3, and A4.

A plurality of contaminant processing devices 1, 2, 3, and 4 may roam along the semiconductor line 3000. In some embodiments, the first contaminant processing device 1 may purify the first zone A1 by sucking in and filtering the chemical gases 50 and particles 60 present in the first zone A1. The second contaminant processing device 2 may purify the second zone A2 by sucking in and filtering the chemical gases 50 and particles 60 present in the second zone A2. The third and fourth contaminant processing devices 3 and 4 can purify the third and fourth zones A3 and A4 by sucking in and filtering the chemical gas 50 and particles 60 present in the third and fourth zones A3 and A4.

In some embodiments, the first contaminant processing device 1 may check the concentration of the chemical gas 50 and the concentration of the particle sensor 123 by using the chemical gas detection sensor 124 and the particle sensor 123 while patrolling within the first zone A1. The second contaminant processing device 2 may check the concentration of the chemical gas 50 and the concentration of the particle sensor 123 by using the chemical gas detection sensor 124 and the particle sensor 123 while patrolling within the second zone A2. The third and fourth contaminant processing devices 3 and 4 may check the concentration of the chemical gas 50 and the concentration of the particle 60 while patrolling within the third zone A3 and the fourth zone A4, respectively.

The first contaminant processing device 1, the second contaminant processing device 2, the third contaminant processing device 3, and the fourth contaminant processing device 4 may communicate with each other.

The controller 1000 may receive the concentration of the chemical gas 50 and the location data of the chemical gas 50 of each zone A1, A2, A3, and A4 from each contaminant processing device 1, 2, 3, and 4.

The controller 1000 may be provided with the concentration of the particles 60 in each zone A1, A2, A3, and A4 and the location data of the particles 60 from each of the contaminant processing devices 1, 2, 3, and 4.

Referring to FIG. 9, the image processing unit 1500 may receive from the controller 1000 the concentration of the chemical gas 50 in each of the zones A1, A2, A3, and A4 and the location data of the chemical gas 50. The image processing unit 1500 may receive from the controller 1000 the concentration of the particles 60 in each zone A1, A2, A3, and A4 and the location data of the particles 60.

The image processing unit 1500 may form a first map 3500 showing the locations of the chemical gas 50 and the particle 60 present in each zone A1, A2, A3, and A4.

The first map 3500 may identify the chemical gas 50 leakage area and the location of the particle 60 present in the semiconductor line 3000. The amount of particles 60 present in each of the zones A1, A2, A3, and A4 may be determined by the first map 3500. The first map 3500 may determine whether chemical gas 50 is leaking from each zone A1, A2, A3, and A4.

The first map 3500 may determine whether more contaminant measurement devices should be deployed. The first map 3500 may allow a human to go in and treat the particles 60 and chemical gas 50.

In some embodiments, each contaminant processing device 1, 2, 3, and 4 may be moved to each zone A1, A2, A3, and A4 of the semiconductor line 3000. For example, the first contaminant processing device 1 may filter chemical gases 50 and particles 60 from the first zone A1 to purify the first zone A1. The first contaminant processing device 1 may measure the chemical gas 50 and the particles 60. Afterwards, the first contaminant processing device 1 may move to the second zone A2 to purify and measure the chemical gas 50 and particles 60.

Semiconductor elements are manufactured through various processes in a clean room equipped with a manufacturing line. During the semiconductor manufacturing process in a clean room, particles or hazardous chemical gases may be emitted. Therefore, the semiconductor line may require a purification process. Conventional particle sensors and chemical gas detection sensors were designed for use in pipes or wall mounting, and thus could not measure and purify in the standby state. Therefore, when particles or chemical gases are generated, they have to be handled and purified by humans.

However, the contaminant processing device according to some embodiments of the present disclosure may include a body unit 110, a purification unit 120, a driving unit 130, a position sensor 115, an image processing unit 1500, and a controller 1000. The contaminant processing device may move around the semiconductor line 3000. The contaminant processing device may constantly measure and purify particles 60 or chemical gases 50 using the purification unit 120.

Specifically, particles 60 and chemical gases 50 may be sucked in by the suction fan 121. The sucked particles 60 and chemical gases 50 may be filtered by the collection unit 122. In this way, the contaminant processing device may constantly measure and purify particles 60 and chemical gases 50 while moving around the semiconductor line 3000. The positions of the particles 60 and chemical gas 50 may be sensed by the position sensor 115. The controller 1000 may be provided with the concentration of the particles 60 and the concentration of the chemical gas 50 from the particle sensor 123 and the chemical gas detection sensor 124. The controller 1000 may be provided with location data of the particles 60 and the chemical gas 50 from the location sensor 115. A first map 3500 showing the concentration and location of particles 60 and the concentration and location of chemical gases 50 may be formed by the image processing unit 1500. The distribution of particles 60 and the concentration of particles 60 may be identified through the first map 3500. The first map 3500 may identify the distribution of the chemical gas 50 and whether the chemical gas 50 is leaking. With reference to the first map 3500, a contaminant processing device may be additionally installed in the semiconductor line 3000 or humans can manually go and treat the particles 60 or chemical gas 50.

As described above, the contaminant processing device may purify the semiconductor line 3000 by constantly moving around the semiconductor line 3000 and filtering particles 60 or chemical gases 50. Additionally, a first map 3500 may be formed to determine the location and concentration of the particles 60 and chemical gas 50 by zones A1, A2, A3, and A4 of the semiconductor line 3000.

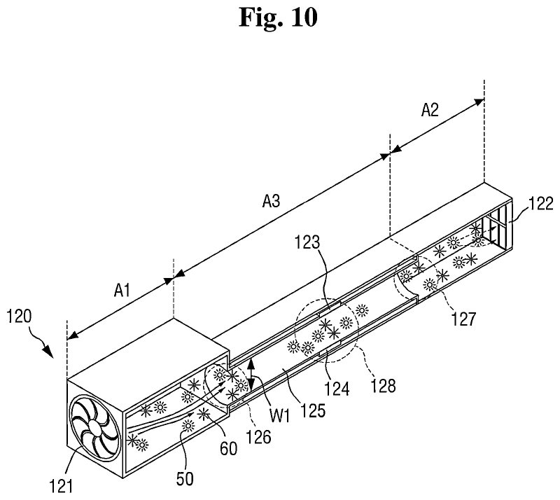

FIG. 10 is a drawing to illustrate a contaminant processing device according to some embodiments of the present disclosure. For convenience of description, duplication of what is described in FIGS. 1 to 5 is briefly described or omitted.

Referring to FIG. 10, the purification unit 120 may include a flow path 125, a suction fan 121, a collection unit 122, a chemical gas detection sensor 124, and a particle sensor 123. The purification unit 120 may include a first area A1, a second area A2, and a third area A3.

The flow path 125 may be installed in the third area A3. The flow path 125 may include one side 126, a center 128, and the other side 127. The width W1 of the flow path 125 may be constant. For example, the width W1 of the flow path 125 may be constant from one side 126 to the center 128 and from the center 128 to the other side 127.

A particle sensor 123 may be installed on an upper portion of the flow path 125. A chemical gas detection sensor 124 may be installed at the lower end of the flow path 125. The particle sensor 123 and the chemical gas detection sensor 124 may be installed in the center 128 but are not limited thereto.

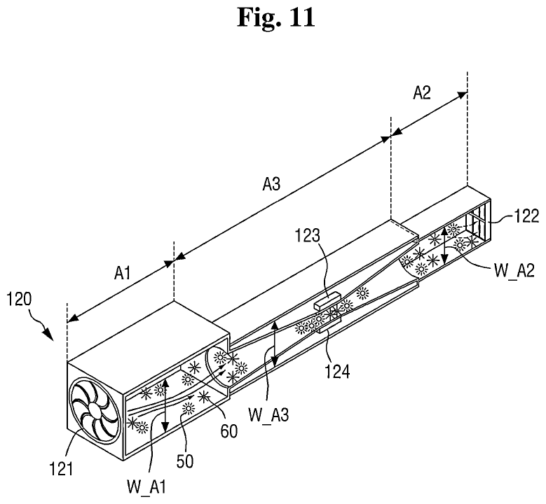

FIG. 11 is a drawing to illustrate a contaminant processing device according to some embodiments of the present disclosure. For convenience of explanation, what is overlapping with what is described in FIGS. 1 to 5 is briefly described or omitted.

Referring to FIG. 11, a purification unit 120 may include a first area A1, a second area A2, and a third area A3. The width may become narrower as it goes from the first area A1, the third area A3, and the second area A2. For example, the width W_A1 of the first area A1 may be larger than the width W_A3 of the third area A3. The width W_A3 of the third area A3 may be larger than the width W_A2 of the second area A2.

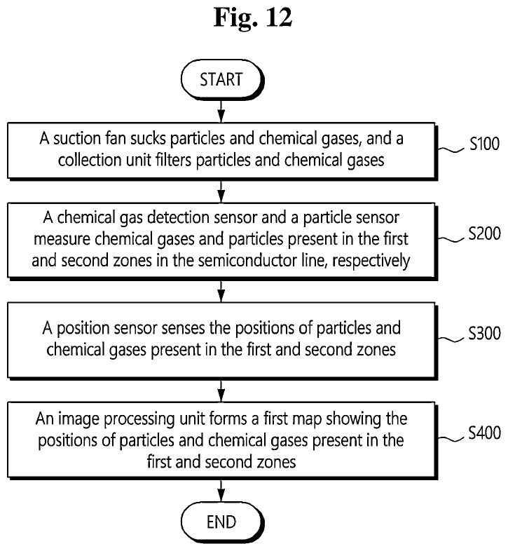

FIG. 12 is a drawing to illustrate a contaminant processing method according to some embodiments of the present disclosure.

A contaminant processing device may be provided including a body unit 110 including different a first surface 111 and a second surface 112, a driving unit 130 installed in the body unit 110 and configured to drive inside the semiconductor line, a position sensor 115 installed in the body unit 110 and configured to sense a position within the semiconductor line, a purification unit 120 installed to penetrate the first surface 111 and the second surface 112, with a flow path 125 disposed between the first surface 111 and the second surface 112, a chemical gas detection sensor 124 and a particle sensor 123 installed in the flow path 125, a suction fan 121 installed in the first area A1 and sucking in chemical gas 50 and particles 60, a purification unit 120 installed in the second area A2 and including a collection unit 122 for filtering the chemical gas 50 and particles 60, a controller 1000 that receives chemical gas concentration and particle concentration from the chemical gas detection sensor 124 and the particle sensor 123, and an image processing unit 1500 that forms a map of the semiconductor lines. (see FIGS. 1 to 5)

Referring to FIG. 12, a suction fan sucks in particles and chemical gas, and a collection unit filters the particles and chemical gas (S100).

The contaminant processing device may be moved around inside the semiconductor line. The particles 60 and chemical gas 50 may be sucked in by the suction fan 121. The sucked particles 60 and chemical gas 50 may be captured by the collection unit 122. In this way, the contaminant processing device may purify the inside of the semiconductor line.

Next, the chemical gas detection sensor and the particle sensor measure the chemical gas and particles present in the first and second zones of the semiconductor line, respectively (S200).

The chemical gas detection sensor 124 and the particle sensor 123 may measure the concentration of the chemical gas 50 and the concentration of the particles 60, respectively. When the suction fan 121 sucks in the chemical gas 50 and the particles 60, the concentration of the chemical gas 50 and the concentration of the particles 60 may be measured by the chemical gas detection sensor 124 and the particle sensor 123. The concentration of the chemical gas 50 and the concentration of the particles 60 may be transmitted to the controller 1000.

Next, the position sensor senses the positions of the particles and chemical gas present in the first and second zones (S300).

When the chemical gas detection sensor 124 and the particles 60 measure the chemical gas 50 and the particles 60, respectively, the positions of the particles 60 and the chemical gas 50 present in the first zone A1 and the second zone A2 in the semiconductor line may be sensed by the position sensor 115. The position of the chemical gas 50 and the position of the particles 60 may be transmitted to the controller 1000.

Subsequently, the image processing unit 1500 forms a first map showing the positions of the particle and the chemical gas present in the first zone and the second zone (S400).

The image processing unit 1500 may receive concentration and location data of the chemical gas 50 from the controller 1000. The image processing unit 1500 may receive concentration and location data of the particles 60 from the controller 1000. Based on the above data, a first map 3500 indicating the location of the chemical gas 50 and the location of the particles 60 may be formed by the image processing unit 1500.

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

What is claimed is:

1. A contaminant processing device comprising:
a body unit including different first surface and second surface;
a driving unit installed in the body unit and configured to drive inside a semiconductor line;
a position sensor installed in the body unit and configured to sense a position within the semiconductor line;
a purification unit installed in the body unit and configured to penetrate the first surface and the second surface, wherein the purification unit includes:
a flow path disposed between the first surface and the second surface,
a chemical gas detection sensor installed in the flow path, measuring a chemical gas entering through the first surface; and
a controller that receives a concentration of the chemical gas from the chemical gas detection sensor, receives position data from the position sensor, and checks the concentration of the chemical gas according to a position within the semiconductor line.

2. The contaminant processing device of claim 1, further comprising a first area disposed between one side of the flow path and the first surface and having a width greater than a maximum width of the flow path, and a second area disposed between the other side of the flow path and the second surface and having a width greater than a maximum width of the flow path.

3. The contaminant processing device of claim 2, further comprising a suction fan installed in the first area and sucking in the chemical gas,
a collection unit installed in the second area and filtering the chemical gas,
wherein the chemical gas detection sensor is disposed between the suction fan and the collection unit.

4. The contaminant processing device of claim 1, wherein the flow path includes one side, a center, and the other side,
a width of the center is smaller than a width of the one side and a width of the other side.

5. The contaminant processing device claim 4, wherein a width of the flow path gradually decreases from the one side to the center, and gradually increases from the center to the other side.

6. The contaminant processing device of claim 5, wherein the chemical gas detection sensor is installed in the center of the flow path.

7. The contaminant processing device of claim 5, wherein the width of the flow path is not constant based on a direction from the one side to the other side,
wherein the chemical gas detection sensor is installed in an area with the smallest width in the flow path.

8. The contaminant processing device of claim 1, further comprising a collection unit installed on the other side of the flow path and filtering the chemical gas,
wherein an exhaust device is installed inside the semiconductor line,
wherein the contaminant processing device moves to the exhaust device and removes the chemical gas collected in the collection unit through the exhaust device.

9. The contaminant processing device of claim 1, wherein the semiconductor line includes different first and second zones,
wherein the first contaminant processing device checks the concentration of the chemical gas using the chemical gas detection sensor while patrolling within the first zone,
wherein the second contaminant processing device communicates with the first contaminant processing device patrolling within the second zone.

10. The contaminant processing device of claim 9, wherein the controller receives the concentration of the chemical gas and a location data of the chemical gas in the first zone from the first contaminant processing device,
and receives the concentration of the chemical gas and a location data of the chemical gas in the second zone from the second contaminant processing device.

11. The contaminant processing device of claim 10, further comprising an image processing unit that receives data on the concentration of the chemical gas and the location of the chemical gas from the controller to form a first map showing the locations of the chemical gas present in the first zone and the second zone.

12. A contaminant processing device comprising:
a purification unit including;
a suction fan installed in a first area and sucking in particles and chemical gases,
a collection unit installed in a second area and filtering the particles and the chemical gases,
a particle sensor and a chemical gas detection sensor installed in a third area between the first area and the second area, wherein the particle sensor measures the particles, and the chemical gas detection sensor measures the chemical gases,
a flow path disposed in the third area,
a body unit connected to the purification unit and including first surface and second surface;
a driving unit installed in the body unit and configured to drive inside the semiconductor line;
a position sensor installed in the body unit and sensing a position within the semiconductor line; and
a controller that receives a concentration of the particles from the particle sensor, receives a concentration of the chemical gas from the chemical gas detection sensor, and checks concentration of the particles and the chemical gas according to the position within the semiconductor line.

13. A contaminant processing device of claim 12, wherein the flow path includes one side, a center, and the other side,
a width of the center is smaller than a width of the one side and a width of the other side,
wherein the particle sensor and the chemical gas detection sensor are installed in a center of the flow path.

14. A contaminant processing device of claim 13, wherein a width of the flow path gradually decreases from the one side to the center, and gradually increases from the center to the other side.

15. A contaminant processing device of claim 12, wherein the first area is disposed between the first surface and one side of the flow path,
wherein the second area is disposed between the second surface and the other side of the flow path,
wherein a width of the first area and the second area is greater than a maximum width of the flow path.

16. A contaminant processing device of claim 12, wherein an exhaust device is installed inside the semiconductor line,
wherein the contaminant processing device moves to the exhaust device to remove the particles and the chemical gas collected in the collection unit through the exhaust device.

17. A contaminant processing device of claim 12, wherein the semiconductor line includes different first zone and second zone,
wherein the first contaminant processing device checks the concentration of the particles and the chemical gas using the particle sensor and the chemical gas detection sensor while patrolling within the first zone,
wherein the second contaminant processing device checks the concentration of the particles and the chemical gas in the second zone and communicates with the first contaminant processing device.

18. A contaminant processing device of claim 17, further comprising an image processing unit that forms a first map showing locations of the particles and the chemical gas present in the first zone and the second zone.

19. A contaminant processing device comprising:
a body unit including different first surface and second surface;
a driving unit installed in the body unit and configured to drive inside a semiconductor line;
a position sensor installed in the body unit and configured to sense a position within the semiconductor line;
a purification unit installed in the body unit and configured to penetrate the first surface and the second surface, wherein the purification includes:
a suction fan installed in a first area and sucking in particles and chemical gases,
a collection unit installed in a second area and filtering the particles and the chemical gases,
a flow path installed in a third area between the first area and the second area,
a particle sensor for measuring the particle and a chemical gas detection sensor for measuring the chemical gas wherein the particle sensor and the chemical gas detection sensor installed in the flow path,
an image processing unit receiving data from the position sensor, the particle sensor, and the chemical gas detection sensor to form a map of the semiconductor line; and
a controller that receives a concentration of the particles from the particle sensor, receives a concentration of the chemical gas from the chemical gas detection sensor, and a position of the particle and a position of the chemical gas from the position sensor,
wherein the flow path includes one side, a center, and the other side, and wherein a width of the flow path gradually decreases from the one side to the center, and gradually increases from the center to the other side,
wherein the semiconductor line includes first zone and second zone,
wherein the particle sensor and the chemical gas detection sensor measure the concentration of the particles and the concentration of the chemical gas present in the first zone and the second zone,
wherein the position sensor senses the positions of the particles and the chemical gas,
wherein the image processing unit forms a first map showing the positions of the particles and the chemical gas present in the first zone and the second zone.

20. A contaminant processing device of claim 19, wherein an exhaust device is installed inside the semiconductor line,
wherein the contaminant processing device moves to the exhaust device to discharge the particles and the chemical gases collected in the collection unit to the outside.
